(19) United States
(12) Patent Application Publication
(10) Pub. No.: US 2024/0252154 A1
(43) Pub. Date: Aug. 1, 2024

(54) BODILY FLUID COLLECTION ASSEMBLY

(71) Applicant: SiPhox, Inc., Burlington, MA (US)

(72) Inventors: Diedrik Rene Vermeulen, Burlington, MA (US); Colin Budries, Burlington, MA (US); Michael Dubrovsky, Burlington (YS); Pavel Korecky, Burlington, MA (US)

(73) Assignee: SiPhox, Inc., Burlington, MA (US)

(21) Appl. No.: 18/427,142

(22) Filed: Jan. 30, 2024

Related U.S. Application Data
(60) Provisional application No. 63/442,002, filed on Jan. 30, 2023.

(51) Int. Cl. A61B 10/00; B01L 3/00; G01N 1/34

(52) U.S. Cl. CPC A61B 10/0045; B01L 3/5023; B01L 3/502715; G01N 1/34; B01L 2200/16; B01L 2300/0636; B01L 2300/069

(57) ABSTRACT

A bodily fluid collection assembly, the assembly includes a collection device including a tube including a chemical lining, a cap, and a fastener wherein the collection device is attached to a multipurpose fluid extraction device including a fluid extraction system, a microfluidic assembly, wherein the microfluidic assembly is configured to provide a flow of the extracted fluid, wherein the microfluidic assembly includes a microfluidic channel, an assay component fluidically connected to the microfluidic assembly, wherein the assay component is configured to test the extracted fluid, and a fluid collecting reservoir fluidically connected to the microfluidic assembly, wherein the fluid collecting reservoir is configured to collect the extracted fluid from the microfluidic assembly.

### CROSS-REFERENCE TO RELATED APPLICATIONS

This application claims the benefit of priority of U.S. Provisional Patent Application Ser. No. 63/442,002, filed on Jan. 30, 2023, and titled “BODILY FLUID COLLECTION TUBE AND CAP,” which is incorporated by reference herein in its entirety.

### FIELD OF THE INVENTION

The present invention generally relates to the field of collection devices. In particular, the present invention is directed to a bodily fluid collection assembly.

### BACKGROUND

Current bodily fluid collection devices serve one or two of the following functions: collection, storage, and dispensing. The ability of a bodily fluid collection device to serve as an appropriate reservoir and dropper remains elusive.

### SUMMARY OF THE DISCLOSURE

In an aspect, a bodily fluid collection assembly, the assembly includes a collection device including a tube including a chemical lining, a cap, and a fastener connecting the cap to the tube wherein the collection device is attached to a multipurpose fluid extraction device including a fluid extraction system, wherein the fluid extraction system is configured to extract a fluid from a user; a microfluidic assembly, wherein the microfluidic assembly is configured to provide a flow of the extracted fluid, wherein the microfluidic assembly includes a microfluidic channel, an assay component fluidically connected to the microfluidic assembly, wherein the assay component is configured to test the extracted fluid, and a fluid collecting reservoir fluidically connected to the microfluidic assembly, wherein the fluid collecting reservoir is configured to collect the extracted fluid from the microfluidic assembly.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIGS. 1A-C are diagrams of an exemplary embodiment of a bodily fluid collection apparatus;

FIG. 2A is an illustration of an exemplary embodiment of an exploded view of an apparatus of a multipurpose fluid extraction device for diagnostics;

FIG. 2B is an illustration of an exemplary embodiment of an assembled view of an apparatus of a multipurpose fluid extraction device for diagnostics;

FIG. 3 is a block diagram of an exemplary workflow of an apparatus of a multipurpose fluid extraction device for diagnostics; and

FIG. 4 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to a bodily fluid collection apparatus integrated in a fluid collection assembly.

Aspects of the present disclosure allow for fluid collection for future testing and fluid assays tests over a photonics sensor that enables digital capture and analysis. In some embodiments, a patient can collect blood for further analysis and getting an immediate initial measurement. In some embodiments, the present disclosure can extract, collect, and test blood in a single application and give the patient the flexibility of in-mail testing but also an immediate test result.

Exemplary embodiments illustrating aspects of the present disclosure are described below in the context of several specific examples.

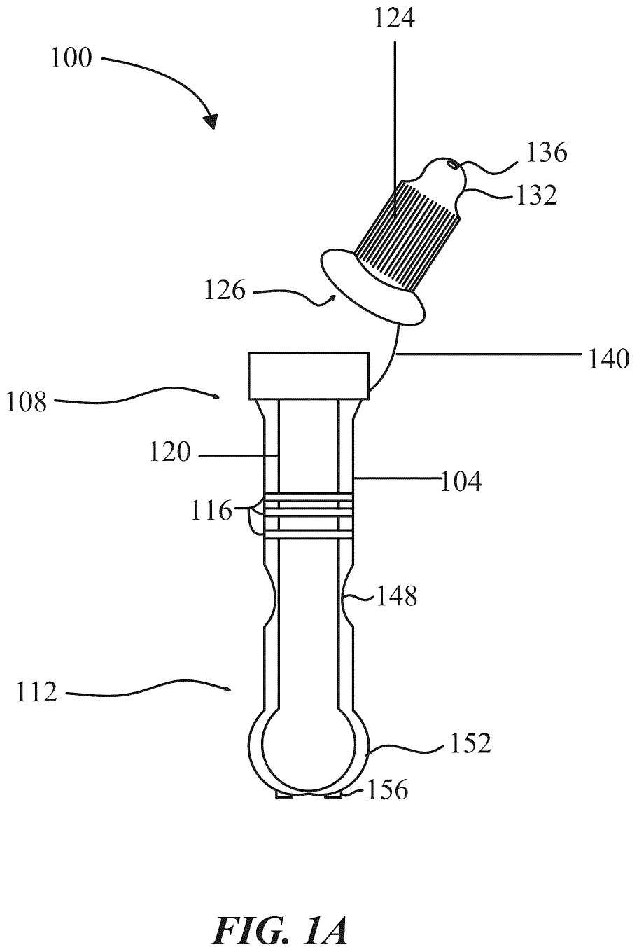

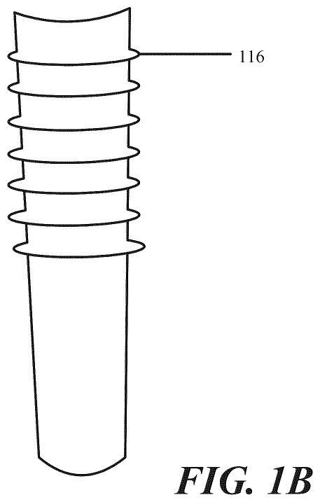

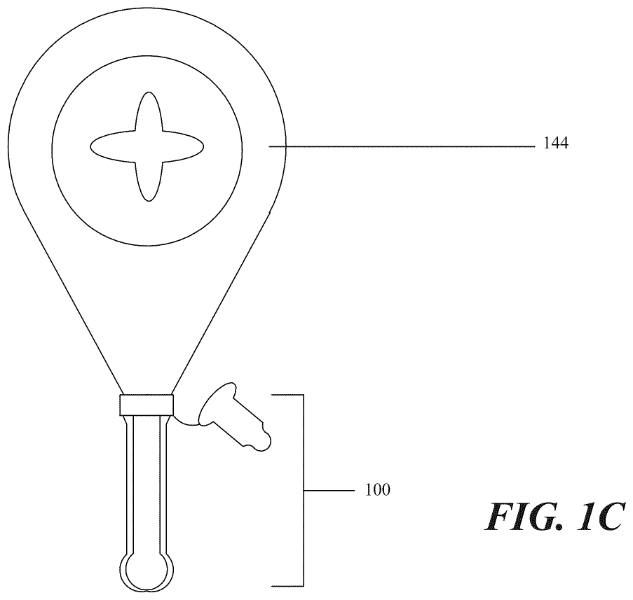

Referring now to FIGS. 1A-C, an exemplary embodiment of a bodily fluid collection apparatus 100 is illustrated. Apparatus includes a tube 104. A “tube,” as used herein is, is a container designed for the collection, storage, and transportation of an object. An object may refer to a biological fluid, specimen, or device. Biological fluids may include blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, as well as cell culture supernatants. Tube 104 may include an outer body also referred as the exterior of tube 104. Tube 104 may be composed of plastic, glass, and the like. For example tube 104 may be made of polyethylene, polystyrene, acrylonitrile butadiene styrene, borosilicate glass, soda-lime glass, or the like. Tube 104 may include an area for labeling where information such as patient details, sample type, and collection date can be recorded. Tube 104 may include temperature-sensitive labels that change color or display indicators based on temperature changes. Tube 104 may be color-coded to indicate the type of additive or treatment inside, helping to identify the tube's purpose. In some embodiments, the proximal end 108 of tube 104 may be firm, enabling tube 104 to be safely ejected from a connection with bodily fluid collection devices as described further below.

A proximal end 108 of tube 104 may be made rigid material. “Rigid material,” as used herein, is material configured to resist deformation and maintain its shape under applied forces. Rigid material may be analyzed based on an elastic modulus. An “elastic modulus,” as used herein, is a measure of a material's stiffness or rigidity. An elastic modulus may include a Young's Modulus (E), Shear Modulus (G), or Bulk Modulus (K). For example, rigid materials may have high values of Young's modulus, indicating high stiffness. In an embodiment wherein proximal end 108 of tube 104 is composed of plastic, ranges for Young's Modulus may include 0.1-4.0 GPa (gigapascals) based on the type of plastic. A distal end 112 of the tube 104 may include a malleable material and is configured to cause ejection of fluid from the tube when the tube is squeezed. “Malleable material,” as used herein, is material configured to be easily deformable using a force applied by a human. Malleable materials may have lower values of Young's modulus, indicating lower stiffness and greater deformability. For example where distal end 112 is made of plastic or rubber, the ranges may include 0.01-1.0 GPa. The distal material of tube 104 may be soft and malleable, enabling the tube 104 to be squeezed, leading to the ejection of the bodily fluid from tube 104. Tube 104 may include plurality of ridges 116 projecting from the outer body. Ridges 116 may include knurling or grip ridges configured to provide a better grip for users when handling the tube 104. “Grip ridges,” as used herein, are textured surfaces located on the exterior of a tube to provide a better grip for handling. Examples of grip ridges include spiral or helical ridges that run along the length of a tube, sections of a tube with indents or raised ribs, ridges designed in a wavy or undulating pattern, and the like. In some embodiments, grip ridges may be made from materials with higher coefficients of friction compared to the rest of tube 104. In some embodiments, grip ridges may include plastic or rubber. Ridges 116 may include an identification ridge. An “identification ridge,” as used herein, is a feature on a tube designed to identify the tube. An identification ridge may include color-coded bands, embossed numbers, and the like configured to distinguish tubes by identifying specific characteristics.

With continued reference to FIGS. 1A-C, tube 104 and/or cap may be made from plastic, plastic may include polyethylene terephthalate, polycarbonate, polyethylene, high-density polyethylene, polyvinyl chloride, poly propylene, poly styrene, bisphenol A, and the like.

Still referring to FIGS. 1A-C, in some embodiments, tube 104 may include removable thermal sleeves or wraps as sleeves may provide a layer of insulation around tube 104, helping to maintain the temperature of the sample. Thermal sleeves or wraps may include material such as fiberglass sleeves, silicone-coated fiberglass, rubber, foam materials, and the like. Tube 104 may include a temperature stabilizing gel to prevent rapid temperature change within tube 104. Temperature-stabilizing gels may include phase-change materials (PCMs), ester-based gels, polymer-based gels, hydrogel composites, salt hydrates and the like. Tube 104 may include foam inserts or padding around the tube 104 to provide insulation. In some embodiments, tube 104 may include a chemical lining 120. A “lining,” as used herein, is a layer applied to an inner surface of a tube. Lining may be configured to interact with or modify the properties of the object/sample contained within tube 104. Chemical lining 120 may be configured for sample preservation. A chemical lining 120 may include a temperature-stabilizing gel, an anticoagulant coating, an anti-foaming agent coating, an anti-clotting agent coating, a barrier coating, a chemical stabilizer, and the like. Anticoagulant coatings may include, ethylenediaminetetraacetic acid, heparin, sodium citrate, Acid-Citrate-Dextrose, any combination thereof and the like. Anti-foaming agent coatings may include polyethylene glycol, ethoxylated sorbitan esters, fatty acid esters, any combination thereof and the like. Anti-clotting agent coatings may include ACD (Acid-Citrate-Dextrose), Citrate-Phosphate-Dextrose-Adenine (CPDA-1), and the like. Barrier coatings may include polymer-based barrier coatings, aluminum barrier coatings, fluoropolymer barrier coatings, and the like. Chemical stabilizers may include antioxidants, chelating agents, emulsifiers, pH stabilizers and the like.

Still referring to FIGS. 1A-C, tube 104 includes a cap 124. A “cap,” as used herein, is a closure configured to seal the opening to a tube 104. Cap 124 enables the storage of an object within the tube 104, preventing the object from leaving the open end of the tube 104. Cap 124 may be composed of material as described above. Cap 124 may include screw caps, snap caps, push-on caps, and the like. Screw caps 116 may include threads on an inner surface of an open end 126 to a cap 124 that match corresponding threads on the outer surface of the tube opening 128. Screw caps may be secured by twisting the cap 124 onto the tube 104. Snap caps may be configured to securely snap onto the opening of the tube 104 without the need for twisting or threading. Snap caps may include a hinge that allows the cap 124 to be easily opened and closed. Push-on caps may be configured to be pushed onto the tube's 104 opening and stay in place without the need for threading or snapping. A “hinge,” as used herein, is a mechanism integrated into a cap that allows for both easy snapping closure and rotational movement. The hinge may include a flexible section of the cap that allows the cap to pivot or rotate relative to the tube. The hinge may include a living hinge, which is a thin, flexible section that acts as a pivot point. In a snap cap embodiment, the cap may be divided into two main sections connected by the hinge. One section may form the main body of the cap, and the other section, which includes the hinge, may be the lid or closure portion. To close the cap, a user may apply a downward pressure on the lid section, causing it to pivot around the hinge and snap shut onto the main body of the cap. The design of cap 124 allows for fluid to be dispensed. When pressure is applied to tube 104, said fluid may flow through a distal portion 132 of the cap outlet 136. The radius of the cap outlet 136 may allow for controlled, precise exit of the liquid. Cap outlet 136 may function similar to a dropper tube and facilitate a controlled and precise dispensing or sampling of liquids. In some embodiments, collection device 104 may function as a dropper tube, wherein fluid may be collected into tube 104 and dropped/dispersed from a cap outlet, as described further below. For example, collection device 100 may be used to collect a blood sample and disperse the blood sample onto a plasma separation card.

Still referring to FIGS. 1A-C, tube 104 includes a fastener. A “fastener,” as used herein, is a component configured to secure a cap 124 to a tube 104. Fastener 140 may extend from cap 124 at its proximal end when attached to the tube 104. In some embodiments, fastener 140 may include a snap mechanism where cap 124 may be designed to snap or click onto tube's 104 opening. In some embodiments, fastener 140 may include ridges 116 or grooves on the inner surface of cap 124 that match corresponding threads on the outer surface of the tube's 104 opening for cap 124 to be twisted onto tube 104.

Still referring to FIGS. 1A-C, in some embodiments, tube 104 may be configured to dispense a fluid onto a bodily fluid testing instrument. A thickness of tube 104 may be based on the type of fluid collected. Tube 104 may include a thick outer body section. Thick tubes provide greater resistance to bending or deformation. In some embodiments, proximal end 108 may be thicker than distal end 112 or the thickest portion of tube 104, such as the outer body of tube 104 at proximal end 108 being the thickest portion. Tube 104 may include a thin outer body section. Thin tubes are more malleable and can be easily shaped or bent. In some embodiments, distal end 112 may be the thinnest portion of tube 104, such as the outer body of tube 104 at distal end 112 being the thinnest portion. For example, proximal end 108 may have a first thickness and distal end 112 may have a second thickness, wherein second thickness is less than first thickness. Still referring to FIGS. 1A-C, the malleable nature of the distal end 112 of tube 104 may enable a force to be applied to the tube 104 which causes the sample to be ejected. The malleable nature can be a result of the material of the tube 104, the shape of the tube 104, a combination of both, or a variety of other forces.

Still referring to FIGS. 1A-C, apparatus 100 may be mechanically attached to a plurality of sample/object collection devices 144. Mechanically attached refers to a connection or attachment between two components or parts that is achieved through mechanical means, involving physical mechanisms or devices 144. Apparatus may be mechanical attached to devices 144 using a thread, screw, or snap mechanism as described above. The ability of tube 104 to connect to fluid collection devices 144 allows it to receive said fluids. In some embodiments, apparatus 100 may be attached to blood collection devices 144. Tube 104 can attach to the end of these devices 144 and serve as a reservoir for body fluid collection. The firm nature of the upper body of tube 104 may allow it to be easily inserted and ejected from the collection device. After tube 104 is removed from the blood collection device, cap 124, while attached using fastener 140, can be placed on top of tube 104 for proper storage. For example, apparatus 100 may be attached to a Tasso Arm Collector. Tube 104 may be pushed into a Tasso microtainer adapter which seals the mechanical connection.

Still referring to FIGS. 1A-C, apparatus 100 may be mechanically attached to a microfluidic assembly as described herein. For example, apparatus 100 may be attached to a biosensing cartridge for storage, transportation, or further assay of a sample. For example, apparatus 104 may be inserted into an outlet channel of a microfluidic assay.

With continued reference to FIGS. 1A-C, in some embodiments, tube 104 may include a grip portion 148. A “grip portion,” for the purposes of this disclosure is a segment of an object that is configured provide enhanced grip to a user. In some embodiments, grip portion 148 may be located at 50% the height of tube 104. In some embodiments, grip portion 148 may be located ⅓ the height of tube 104. In some embodiments, grip portion 148 may be located ⅔ the height of tube 104. In some embodiments, grip portion 148 may include grip ridges as described above. In some embodiments, grip portion 148 may include knurling as described above. In some embodiments, grip portion 148 may include an abrasive surface to increase grip. In some embodiments, grip portion 148 may include a contoured shape configured to make tube 104 easier to grip. As a non-limiting example, grip portion 148 may include one or more indentations. The indentations may be configured to accommodate the fingers of a holder. In some embodiments, indentations may include a knurled and/or abrasive surface as described above. In some embodiments, grip portion 148 may take up 10 to 70% of the height of tube 104. In some embodiments, grip portion 148 may take up 30 to 60% of the height of tube 104. In some embodiments, grip portion 148 may take up 40 to 50% of the height of tube 104. In some embodiments, grip portion 148 may take up 10% of the height of tube 104. In some embodiments, grip portion 148 may take up 20% of the height of tube 104. In some embodiments, grip portion 148 may take up 30% of the height of tube 104.

With continued reference to FIGS. 1A-C, in some embodiments, tube 104 may include a bulb 152. A “bulb,” for the purposes of this disclosure, is a portion of the tube that has a greater diameter relative to the adjacent portions of the tube. In some embodiments, diameter of bulb 152 may be 20% larger than the rest of the tube 104. In some embodiments, bulb 152 may be configured to be squeezed in order to expel material from the tube.

With continued reference to FIGS. 1A-C, in some embodiments, distal part 112 of tube 104 may include bulb 156. In some embodiments, distal part 112 of tube 104 may be bulb 156. In some embodiments, distal part 112 of tube 104 may include grip portion 148. In some embodiments, grip portion 148 may include a thinner thickness of tube 104 sidewall so that it is easier to squeeze tube 104 at grip portion 148.

With continued reference to FIGS. 1A-C, in some embodiments, tube 104 may include one or more legs 156. A “leg,” for the purposes of this disclosure, is a support structure of a device, configured to allow the device to stand freely. In some embodiments, tube 104 may include one leg 156. In some embodiments, tube 104 may include two legs 156. In some embodiments, tube 104 may include three legs 156. In some embodiments, tube 104 may include four legs 156. In some embodiments, tube 104 may include five legs 156. In some embodiments, legs 156 may be disposed radially about the central axis of tube 104 at regular intervals. In some embodiments, legs 156 may be made from the same material as the tube. Legs 156 may include any plastic as disclosed above. In some embodiments, legs 156 may include a rubber coating or layer in order to increase its gripping capability. In some embodiments, legs 156 may include rubber.

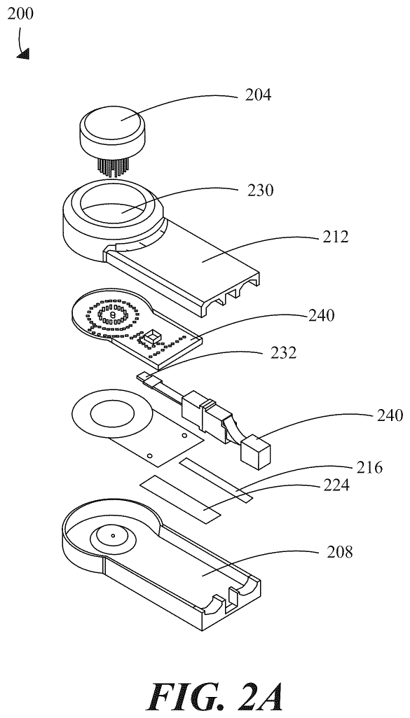

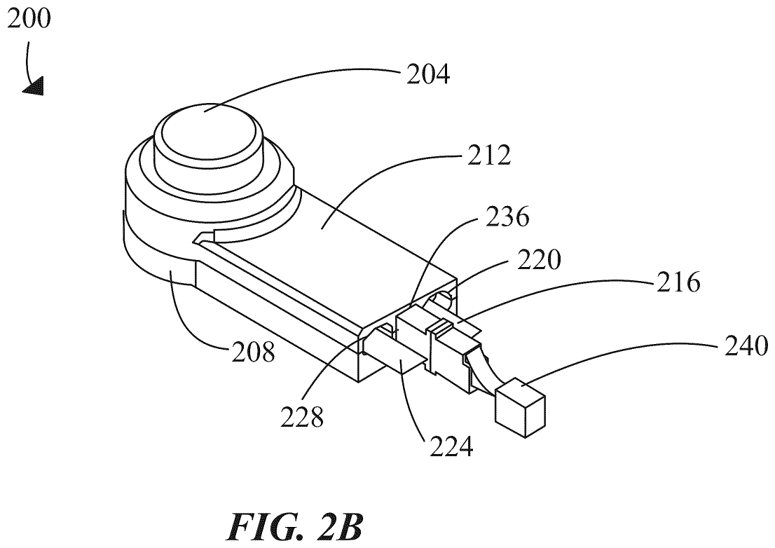

Referring now to FIGS. 2A-B, FIG. 2A illustrates an exemplary embodiment of an exploded view of an apparatus 200 of a fluid extraction device 204 for diagnostics. Apparatus 100 may be mechanically attached or integrated into apparatus 200. FIG. 2B illustrates an exemplary embodiment of an assembled view of the apparatus 200 of the fluid extraction device 204 for diagnostics. In some embodiments, the apparatus 200 may be reusable. In some embodiments, the apparatus 200 may include a housing. As used in this disclosure, a “housing” refers to an outer structure configured to contain a plurality of components. In some embodiments, this may include, without limitation, components of apparatus 200 as described in this disclosure. In some embodiments, the housing may be portable. For the purposes of this disclosure, a “portable” refers to the capability for an object to be easily carried or moved from place to place by a person. A housing may be configured to encapsulate at least a portion of fluid extraction device 204, wherein the housing is portable. In some cases, the housing may include a durable, lightweight material such as without limitation, plastic, metal, and/or the like. In some embodiments, the housing may be designed and configured to protect sensitive components of apparatus 200 from damage or contamination. In some embodiments, the housing may include one or more physical notches and/or grooves that allow for precise placement of devices and/or components. In some embodiments, the housing may include one or more optical markers or alignment indicators that are visible (through human eye, microscope, any other imaging system, and/or the like) and allow for accurate positioning of devices and/or components. In some embodiments, the housing may include one or more surface coatings and/or modifications that reduce the likelihood of unwanted adhesion or interference with external components and/or substances. Additionally, or alternatively, the housing may further include features such as latches, clips, or other fasteners that help to secure apparatus 200 in place during use.

With continued reference to FIGS. 2A-B, in some embodiments, a housing may include a first housing 208 and a second housing 212 as shown in FIGS. 2A-B. As a non-limiting example, the second housing 212 may be placed on atop of the first housing 208. The first housing 208 and the second housing 212 may allow the housing to be assembled or disassembled easily. In an embodiment, the first housing 208 and the second housing 212 may be matched permanently. As a non-limiting example, the first housing and the second housing 212 may be permanently connected to each other using a variety of techniques, such as but not limited to welding, soldering, brazing, adhesive bonding, or mechanical fasteners. In another embodiment, the first housing 208 and the second housing 212 may be matched temporarily. In another word, the first housing 208 and the second housing 212 may be removably connected to each other. As a non-limiting example, the first housing 208 and the second housing 212 may be removably connected to each other using a mechanical fastener. Mechanical fasteners may include bolts, screws, nuts, washers, rivets, pins, and the like. For the purposes of this disclosure, “removably connected” refers to an ability for an object that is connected to another object to be disconnected from the other object without damaging or breaking said objects. In some embodiments, the removable connection may include threaded connection. For the purposes of this disclosure, “threaded connection” is a type of connection that involves mating male and female halves together to create a connection to hold the threads together. As a non-limiting example, the threaded connection may be done by way of gendered mating components. As a non-limiting example, the gendered mating components may include a male component or plug which is inserted within a female component or socket. In some cases, the threaded connection may be removable. In some cases, the threaded connection may be removable, but requires a specialized tool or key for removal. In some embodiments, the threaded connection may be achieved by way of one or more of plug and socket mates, pogo pin contact, crown spring mates, and the like. In some cases, the threaded connection may be keyed to ensure proper alignment of a mating component. In some cases, the threaded connection may be lockable. As used in this disclosure, a “mating component” is a component that mates with at least another component. As a non-limiting example, the mating component may include a mechanical connector. In another embodiment, the removable connection may include bayonet connections. The bayonet connections use a locking mechanism that allows the two components to be connected by inserting and twisting them into place. In another embodiment, the removable connection may include snap-fit connections. In some embodiments, the snap-fit connections may include a series of tabs or hooks that snap into place when the two components are pushed together. As a non-limiting example, the snap-fit connections may include snap-fit clips, snap-fit tabs, snap-fit hinges, snap-fit latches, snap-fit hooks, snap-fit pins, and the like. In another embodiment, the removable connection may include latch connections. The latch connections use a latch or locking mechanism that secures the two components together. As a non-limiting example, the latch connections may include cabinet latches, door latches, aircraft fasteners, and the like. In another embodiment, the removable connection may include clamp connections. In some embodiments, the clamp connections use a clamp or compression mechanism to hold the two components together. As a non-limiting example, the clamp connections may include hose clamps, c-clamps, pipe clamps, wire rope clamps, shaft collars, spring clamps, and the like. In another embodiment, the removable connection may include magnetic connections. In some embodiments, the magnetic connections use magnets to hold the two components together. In some embodiments, the removable connection may include connectors, screws, adapters, feedthrough, and the like. For the purposes of this disclosure, a “connector” is a component configured to create an electrical or mechanical connection between two or more objects. Examples of connectors include plug and socket connectors, terminal blocks, crimp connectors, and the like.

With continued reference to FIGS. 2A-B, in some embodiments, a housing may include at least an aperture that provides a path for a connection between components of an apparatus 200 for communication. For the purposes of this disclosure, an “aperture” is an opening or hole through which something passes or can be seen. As a non-limiting example, an assay component 216 may be removably inserted in the housing using a first aperture 220 of the housing. For example, and without limitation, the assay component 216 may be removably inserted into the housing through the first aperture 220 for testing at least a fluid and removed once the testing is finished. As another non-limiting example, a fluid collecting reservoir 224 may be removably inserted in the housing using a second aperture 228. For example, and without limitation, the fluid collecting reservoir 224 may be removably inserted into the housing through the second aperture 228 for collecting the at least a fluid and removed through the second aperture 228 once the at least a fluid is collected. In some embodiments, tube 104 may be removably inserted into either first aperture 220 or second aperture 228 for collecting the at least a fluid. For example, tube 104 may be inserted into second aperture 228 instead of fluid collecting reservoir 224. Tube 104 may be inserted into the housing after assays have been performed as a final collection step. For the purposes of this disclosure, “removably inserted” refers to an object that has been inserted or placed into another object such that the object can be removed from the other object without causing damage or leaving any residue behind. As another non-limiting example, a fluid extraction system 204 may be removably connected to the housing using a third aperture 230 of the housing. For example, and without limitation, the fluid extraction system 204 may be removably connected to the third aperture 230 of the housing using a snap-fit connections as described above. The assay component 216, the fluid collecting reservoir 224 and the fluid extraction system 204 disclosed herein are described further in detail below. As another non-limiting example, a photonic sensor 232 may be removably inserted into the housing using a fourth aperture 236 of the housing. As another non-limiting example, the photonic sensor 232 may be removably connected into the housing using the fourth aperture 236 of the housing using a snap-fit connection as described above. As another non-limiting example, the photonic sensor 232 may be removed from the housing when a first housing 208 and a second housing 212 get disassembled.

With continued reference to FIGS. 2A-B, in some embodiments, a housing may include one or more bumps inside the housing that allow for precise placement of devices and/or components. As a non-limiting example, one or more bumps may be present to indicate the extent to which a fluid collecting reservoir 224 and/or an assay component 216 can be removably inserted into the housing through at least an aperture, such as a first aperture 220 and a second aperture 228. When, for example, and without limitation, the fluid collecting reservoir 224 and/or assay component 216 is being inserted into the housing through at least one aperture, the one or more bumps may prevent further placement into the housing. In some embodiments, the housing may include one or more optical markers or alignment indicators that are visible (through human eye, microscope, any other imaging system, and/or the like) and allow for accurate positioning of devices and/or components, such as but not limited to the fluid collecting reservoir 224, the assay component 216 and/or a fluid extraction system 204. In some embodiments, tube 104, the fluid collecting reservoir 224, the assay component 216 and/or the fluid extraction system 204 may include the one or more optical markers or alignment indicators that are visible (through human eye, microscope, any other imaging system, and/or the like) and allow for accurate positioning of them into the housing.

With continued reference to FIGS. 2A-B, as used in this disclosure, “communication” is an attribute wherein two or more relata interact with one another, for example within a specific domain or in a certain manner. In some cases, communication between two or more relata may be of a specific domain, such as without limitation electric communication, fluidic communication, informatic communication, mechanical communication, and the like. As used in this disclosure, “informatic communication” is an attribute wherein two or more relata interact with one another by way of an information flow or information in general. For example, and without limitation, a communication between a photonic sensor 232 and a reader device 240 may include the informatic communication. The photonic sensor 232 and the reader device 240 disclosed herein are described in detail below. As used in this disclosure, “mechanic communication” is an attribute wherein two or more relata interact with one another by way of mechanical means, for instance mechanical effort (e.g., force) and flow (e.g., velocity). “Electric communication,” as used in this disclosure, is an attribute wherein two or more relata interact with one another by way of an electric current or electricity in general. “Fluidic communication,” as used in this disclosure, is an attribute wherein two or more relata interact with one another by way of a fluidic flow or fluid in general. For example, and without limitation, a communication between a microfluidic assembly 240 and a fluid collecting reservoir 224 may include the fluidic communication. For example, and without limitation, a communication between the microfluidic assembly 240 and an assay component 216 may include the fluidic communication. For example, and without limitation, a communication between the microfluidic assembly 240 and a fluid extraction system 204 may include the fluidic communication.

With continued reference to FIGS. 2A-B, an apparatus 200 includes a fluid extraction system 204. For the purposes of this disclosure, a “fluid extraction system” is a system that is configured to extract a fluid from a user. For the purposes of this disclosure, a “user” is any human or animal. In some embodiments, the “user” may be consistent with a “patient” In some embodiments, the fluid extraction system 204 may be disposable after use. In some embodiments, the fluid extraction system 204 may be replaceable. For the purposes of this disclosure, “fluid” is any sample that has no fixed shape and yields easily to external pressure. For the purposes of this disclosure, a “sample” is some quantity of tissue, fluid, or the like extracted from a subject organism, such as without limitation, a human being, and/or a substance derived therefrom. As a non-limiting example, the fluid may include cerebrospinal fluid, whole blood, urine samples, and the like. In some embodiments, the fluid may include one or more analytes. For the purposes of this disclosure, an “analyte” is a substance that is of interest in an analytical procedure. As a non-limiting example, the one or more analytes may include glucose, proteins, hormones, antibodies, and the like. As another non-limiting example, the one or more analytes may include Albumin, C-reactive protein, SARS-COV-2 protein, Thyroxine-binding globulin, Thyroxine-binding prealbumin (transthyretin), Ceruloplasmin, Haptoglobin, Apolipoprotein A-I (protein of HDL), Apolipoprotein A-II (another protein of HDL), Apolipoprotein B-200 (protein of LDL), Transferrin, Serum free light chains (info from LabCorp), Antithrombin III, Fibrinogen, Lysozyme, Plasminogen, C3 complement, C4 complement, D-dimer, a1-Fetoprotein (AFP), a2-Macroglobulin (AMG), Retinol binding protein, Alpha1-Antitrypsin (A1AT or AAT), a1-Acid Glycoprotein (or orosomucoid), cx1-antichymotrypsin (Serpin family A member 3) ghrelin, Hemopexin, Complement factor H, Vitronectin, C4b binding protein (Complement component 4 binding protein beta), Cysteine rich secretory glycoprotein LCCL domain containing 2 (Crispld2), Complement C5, Alpha 1-B glycoprotein, Apolipoprotein H, Apolipoprotein A4, Plasminogen, GC vitamin D binding protein (DBP), Histidine rich glycoprotein, Coagulation factor II, thrombin, Glycosylphosphatidylinositol specific phospholipase D1, Complement C1s, Fetuin B, Kininogen 1, Complement C9, Gelsolin, Apolipoprotein C3, Serpin family A member 6, Apolipoprotein C1, Paraoxonase 1, Serum amyloid 4, Alpha-2 glycoprotein 1, zinc-binding, Afamin, Apolipoprotein C2, Clusterin, Apolipoprotein E, Serpin family A member 7, Complement component 4 binding protein alpha, Kallikrein B1, Amyloid P component, Renalase, FAD dependent amine oxidase, Thrombospondin 1, Leucine rich alpha-2 glycoprotein 1, Lipopolysaccharide binding protein, Protein S, Retinal binding protein 4, Apolipoprotein F, Ficolin 3, Phospholipase transfer protein, Serpin family F member 1, Adiponectin, C1Q and collagen domain, Insulin such as growth factor binding acid labile subunit, Ficolin 2, Hyaluronan binding protein 2, Mannan binding lectin serine peptidase 1, C-type lectin domain family 3 member B, Coagulation factor V, Complement C1r subcomponent, Lecithin-cholesterol acyltransferase, CDS molecule, Serpin family A member 10, Apolipoprotein L1, Insulin like growth factor binding protein 3, Cholesterol ester transfer protein, CD14, Glutathione peroxidase 3, CD263, Paraoxanase 3, Protein Z, Ficolin 1, Transferrin receptor, ADAM metallopeptidase with thrombospondin type 1 motif 13, Complement factor D, Cystatin C, Apolipoprotein C4, Myeloperoxidase, Mannose binding lectin 2, Complement factor B, C-C motif chemokine ligand 28, Tenascin C, Vascular cell adhesion molecule 1 (VCAM1), Cathelicidin antimicrobial peptide, Insulin like growth factor binding protein 2, Complement factor H related 3, Insulin like growth factor 2, Complement C1q C chain, Mannan binding lectin serine peptidase 2, Lipase G, C1q and TNF related 9, Fibrinogen alpha chain, C1q and TNF related 6, Von Willebrand factor, Gremlin 1, C1q and TNF related 5, C1q and TNF related 1, Serum amyloid A1, Angiogenin, C1q and TNF related 7, Orosomucoid 2, Angiopoietin like 3, Fc receptor like BMP4, Chromogranin A, and the like. Persons skilled in the art, upon reviewing the entirety of this disclosure, may appreciate various analytes that may be used for an apparatus 200. Additional disclosures related to the at least an analyte may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to the FIGS. 2A-B, in some embodiments, a fluid extraction system 204 may include an optical skin piercing component. For the purposes of this disclosure, an “optical skin piercing component” is a device that uses light-based technology to pierce the skin. In some embodiments, the optical skin piercing component may include a light source to create a beam of light that can be focused on a specific area of the skin to create a hole or puncture. In some embodiments, the light source may produce a narrow, focused beam of light that can be precisely controlled in terms of wavelength, pulse duration, size and shape of the beam, and intensity. As a non-limiting example, the optical skin piercing component may include a laser lancet. In some embodiments, the laser lancets may emit a focused beam of light that heats the skin at a specific location, causing it to vaporize and create a small incision. In some embodiments, the laser beam may be controlled by a computing device. The computing device disclosed herein may be consistent with any computing device disclosed in the entirety of this disclosure. As a non-limiting example, the computing device may control over the size and depth of the incision. In some embodiments, the optical skin piercing component may be actuated using an actuator described below. As a non-limiting example, a user may push a button to actuate the optical skin piercing component.

With continued reference to the FIGS. 2A-B, in another embodiment, the fluid extraction system 204 may include a mechanical skin piercing component. As a non-limiting example, the mechanical skin piercing component may include microneedles, micro blades, and the like as shown in FIGS. 2A-B. As another non-limiting example, the mechanical skin piercing component may include a lancet. In some embodiments, the mechanical skin piercing component may include stainless steel or other metals. In some embodiments, the mechanical skin piercing component may include various sizes and shapes. In an embodiment, the mechanical skin piercing component may be manually operated. As a non-limiting example, a user may manually make an incision on the skin using the mechanical skin piercing component. As another non-limiting example, the user may manually make an incision on the skin by pushing an actuator, such as but not limited to a button, to actuate the mechanical skin piercing component. In another embodiment, the mechanical skin piercing component may be automated. As a non-limiting example, the mechanical skin piercing component may be automated using a computing device. For example, and without limitation, the computing device may be implemented in a reader device 240. In an embodiment, the mechanical skin piercing component can be used for single or multiple punctures.

With continued reference to the FIGS. 2A-B, in some embodiments, the fluid extraction system 204 may include an actuator. For the purposes of this disclosure, an “actuator” is a component of a device that is responsible for moving and/or controlling a mechanism or system. As a non-limiting example, the actuator may include a button, lever, and the like. In some embodiments, the actuator may be pressed or clicked to perform a specific action. As a non-limiting example, the actuator may be configured to actuate the optical skin piercing component of a fluid extraction system 204. As another non-limiting example, the actuator may be configured to actuate a mechanical skin piercing component of the fluid extraction system 204. In some embodiments, the actuator may be controlled by a computing device.

With continued reference to FIGS. 2A-B, an actuator may include a component of a machine that is responsible for moving and/or controlling a mechanism or system. An actuator may in some cases, require a control signal and/or a source of energy or power. In some cases, a control signal may be relatively low energy. Exemplary control signal forms include electric potential or current, pneumatic pressure or flow, or hydraulic fluid pressure or flow, mechanical force/torque or velocity, or even human power. In some cases, an actuator may have an energy or power source other than control signal. This may include a main energy source, which may include for example electric power, hydraulic power, pneumatic power, mechanical power, and the like. In some cases, upon receiving a control signal, an actuator responds by converting source power into mechanical motion. In some cases, an actuator may be understood as a form of automation or automatic control.

With continued reference to FIGS. 2A-B, in some embodiments, actuator may include a hydraulic actuator. A hydraulic actuator may consist of a cylinder or fluid motor that uses hydraulic power to facilitate mechanical operation. Output of hydraulic actuator may include mechanical motion, such as without limitation linear, rotatory, or oscillatory motion. In some cases, hydraulic actuator may employ a liquid hydraulic fluid. As liquids, in some cases. are incompressible, a hydraulic actuator can exert large forces. Additionally, as force is equal to pressure multiplied by area, hydraulic actuators may act as force transformers with changes in area (e.g., cross sectional area of cylinder and/or piston). An exemplary hydraulic cylinder may consist of a hollow cylindrical tube within which a piston can slide. In some cases, a hydraulic cylinder may be considered single acting. Single acting may be used when fluid pressure is applied substantially to just one side of a piston. Consequently, a single acting piston can move in only one direction. In some cases, a spring may be used to give a single acting piston a return stroke. In some cases, a hydraulic cylinder may be double acting. Double acting may be used when pressure is applied substantially on each side of a piston; any difference in resultant force between the two sides of the piston causes the piston to move.

With continued reference to FIGS. 2A-B, in some embodiments, actuator may include a pneumatic actuator. In some cases, a pneumatic actuator may enable considerable forces to be produced from relatively small changes in gas pressure. In some cases, a pneumatic actuator may respond more quickly than other types of actuators, for example hydraulic actuators. A pneumatic actuator may use compressible fluid (e.g., air). In some cases, a pneumatic actuator may operate on compressed air. Operation of hydraulic and/or pneumatic actuators may include control of one or more valves, circuits, fluid pumps, and/or fluid manifolds.

With continued reference to FIGS. 2A-B, in some cases, actuator may include an electric actuator. Electric actuator may include any electromechanical actuators, linear motors, and the like. In some cases, actuator may include an electromechanical actuator. An electromechanical actuator may convert a rotational force of an electric rotary motor into a linear movement to generate a linear movement through a mechanism. Exemplary mechanisms, include rotational to translational motion transformers, such as without limitation a belt, a screw, a crank, a cam, a linkage, a scotch yoke, and the like. In some cases, control of an electromechanical actuator may include control of electric motor, for instance a control signal may control one or more electric motor parameters to control electromechanical actuator. Exemplary non-limitation electric motor parameters include rotational position, input torque, velocity, current, and potential. electric actuator may include a linear motor. Linear motors may differ from electromechanical actuators, as power from linear motors is output directly as translational motion, rather than output as rotational motion and converted to translational motion. In some cases, a linear motor may cause lower friction losses than other devices. Linear motors may be further specified into at least 3 different categories, including flat linear motor, U-channel linear motors and tubular linear motors. Linear motors may be directly controlled by a control signal for controlling one or more linear motor parameters. Exemplary linear motor parameters include without limitation position, force, velocity, potential, and current.

With continued reference to FIGS. 2A-B, in some embodiments, an actuator may include a mechanical actuator. In some cases, a mechanical actuator may function to execute movement by converting one kind of motion, such as rotary motion, into another kind, such as linear motion. An exemplary mechanical actuator includes a rack and pinion. In some cases, a mechanical power source, such as a power take off may serve as power source for a mechanical actuator. Mechanical actuators may employ any number of mechanism, including for example without limitation gears, rails, pulleys, cables, linkages, and the like.

With continued reference to the FIGS. 2A-B, an apparatus 200 includes a microfluidic assembly 240. For the purposes of this disclosure, a “microfluidic assembly” is an assembly that is configured to act upon fluids at a small scale, such as without limitation a sub-millimeter scale. At small scales, surface forces may dominate volumetric forces. In some embodiments, the microfluidic assembly 240 is configured to provide a flow of extracted fluid. In some embodiments, plasma may be separated in the microfluidic assembly 240. For the purposes of this disclosure, “extracted fluid” is a fluid that is extracted from the skin using a fluid extraction system. In some embodiments, the microfluidic assembly 240 may be configured to provide the flow of the extracted fluid over a photonic sensor 232. As a non-limiting example, the microfluidic assembly 240 may be on atop one or more resonators of the photonic sensor 232. The photonic sensor 232 and the one or more resonators disclosed herein are further described below. Additional disclosure related to the microfluidic assembly 240 may be found in U.S. patent application Ser. No. 18/221,712, filed on Mar. 15, 2023, entitled “APPARATUS AND METHODS FOR PERFORMING MICROFLUIDIC-BASED BIOCHEMICAL ASSAYS,” and in U.S. patent application Ser. No. 17/859,932, filed on Jul. 7, 2022, entitled “SYSTEM AND METHODS FOR FLUID SENSING USING PASSIVE FLOW,” the entirety of which are incorporated herein by references.

With continued reference to FIGS. 2A-B, in some embodiments, a microfluidic assembly 240 includes a microfluidic channel. As used in this disclosure, a “microfluidic channel” is a structure within microfluidic assembly that is designed and/or configured to manipulate one or more fluids at micro scale. In some cases, microfluidic channel may enable a precise manipulation of fluids and samples in a controlled and/or reproducible manner within microfluidic assembly 240. In some embodiments, microfluidic channel of microfluidic assembly 240 may be designed and arranged based on particular needs. In other embodiments, the microfluidic channel of microfluidic assembly 240 may be varied depending on the type of a fluid being used, that is directly contact with microfluidic channel. In a non-limiting example, attributes of the microfluidic channel such as, without the size and/or shape of the substrate may be determined as a function of specific assay protocols.

With continued reference to FIGS. 2A-B, in some embodiments, a microfluidic assembly 240 may further include at least a flow component connected with at least a microfluidic channel configured to flow at least a fluid through a photonic sensor 232. In some embodiments, at least a flow component may include a passive flow component configured to initiate a passive flow process. In some embodiments, the passive flow component may be in fluidic communication with a fluid collecting reservoir 224 or lub 104. The fluid collecting reservoir 224 disclosed herein is further described below. As a non-limiting example, the fluid collecting reservoir 224 or tub 104 may be configured to passively pump the extracted fluid for the flow of the extracted fluid of the microfluidic assembly 240. For the purposes of this disclosure, “passively pump” refers to pumping a fluid without an external actuator or power source. For example, and without limitation, the microfluidic assembly 240 and the fluid collecting reservoir 224 or tube 104 may create a concentration gradient that drives the flow of fluids through the microfluidic assembly 240 (e.g., osmosis). In some embodiments, the passive flow component may be in fluidic communication with an assay component 216. As used in this disclosure, a “passive flow component” is a component, typically of a microfluidic assembly, that imparts a passive flow on at least a fluid, wherein the “passive flow,” for the purpose of this disclosure, is flow of the at least a fluid, which is induced absent any external actuators, fields, or power sources. As used in this disclosure, a “passive flow process” is a plurality of actions or steps taken on passive flow component in order to impart a passive flow on at least a fluid. In some embodiments, the passive flow component may employ one or more passive flow techniques in order to initiate passive flow process; for instance, and without limitation, passive flow techniques may include osmosis, capillary action, surface tension, pressure, gravity-driven flow, hydrostatic flow, vacuums, and the like. As a non-limiting example, the capillary action can occur when a fluid flows through a microfluidic channel due to the adhesive and cohesive properties of the fluid. When the fluid encounters a surface, such as the walls of the microfluidic channel, the fluid can be drawn into the microfluidic channel by the capillary action. The fluid can then be transported through the microfluidic channel by the combined forces of adhesion and cohesion, which cause the fluid to flow along the microfluidic channel's surface. As another non-limiting example, the gravity-driven flow may allow the fluid to flow downhill due to the force of gravity. In a non-limiting example, the passive flow component may be consistent with any passive flow component described in U.S. patent application Ser. No. 18/221,712, filed on Mar. 15, 2023, entitled “APPARATUS AND METHODS FOR PERFORMING MICROFLUIDIC-BASED BIOCHEMICAL ASSAYS,” and in U.S. patent application Ser. No. 17/859,932, filed on Jul. 7, 2022, entitled “SYSTEM AND METHODS FOR FLUID SENSING USING PASSIVE FLOW,” the entirety of which are incorporated herein by references.

With continued reference to FIGS. 2A-B, in other embodiments, at least a flow component may include an active flow component configured to initiate an active flow process. As used in this disclosure, an “active flow component” is a component that imparts an active flow on at least a fluid, wherein the “active flow,” for the purpose of this disclosure, is flow of the at least a fluid which is induced by external actuators, fields, or power sources. As used in this disclosure, an “active flow process” is a plurality of actions or steps taken on active flow component in order to impart active flow on at least a fluid. In some embodiments, the active flow component is in fluidic communication with a fluid collecting reservoir 224 or tube. In some embodiments, the active flow component may be in fluidic communication with an assay component 216. In a non-limiting example, the active flow component may include one or more pumps. The one or more pumps may include a substantially constant pressure pump (e.g., centrifugal pump) or a substantially constant flow pump (e.g., positive displacement pump, gear pump, and the like). The one or more pumps can be hydrostatic or hydrodynamic. As used in this disclosure, a “pump” is a mechanical source of power that converts mechanical power into fluidic energy. The one or more pumps may generate flow with enough power to overcome pressure induced by a load at a pump outlet. The one or more pumps may generate a vacuum at a pump inlet, thereby forcing fluid into the pump inlet to the one or more pumps pump and by mechanical action delivering the fluid to a pump outlet. The hydrostatic pumps may include positive displacement pumps. The hydrodynamic pumps can be fixed displacement pumps, in which displacement may not be adjusted, or variable displacement pumps, in which the displacement may be adjusted. Exemplary non-limiting pumps include gear pumps, rotary vane pumps, screw pumps, bent axis pumps, inline axial piston pumps, radial piston pumps, and the like. The one or more pumps may be powered by any rotational mechanical work source, for example without limitation and electric motor or a power take off from an engine. The one or more pumps may be in fluidic communication with the fluid collecting reservoir 224 or tube 104.

With continued reference to FIGS. 2A-B, in some embodiments, a microfluidic assembly 240 may include multiple layers of a microfluid. As a non-limiting example, the multiple layers of the microfluidic channel may be created by multilayering Pressure Sensitive adhesive (PSA) over etched elements on the piece or other PSA laminates. In some embodiments, the microfluidic channel of the microfluidic assembly 240 may be sealed with adhesive laminate. In some embodiments, the microfluidic channel of the microfluidic assembly may be formed by various techniques such as but not limited to photolithography, etching, deposition, and/or other microfabrication techniques. As a non-limiting example, the microfluidic channel may be etched on a substrate such as but not limited to glass, silicon, or polymers. In some embodiments, the microfluidic channels of the microfluidic assembly 240 may include various sizes depending on capillary needs as well as characteristics of fluids. In some embodiments, the microfluidic assembly may be configured to drive the fluid over a photonic sensor 232 and then to an assay component 216 and/or a fluid collecting reservoir 224. An exemplary configuration of the flow of the fluid is illustrated in FIG. 2.

With continued reference to FIGS. 2A-B, in some embodiments, a microfluidic assembly may include an amplifier. As used in this disclosure, an “amplifier” is a component that can increase the sensitivity of the system to detect specific analytes. For an amplified binding, the amplifiers can be placed on microfluidic channels to later be driven alongside the extracted fluid onto a photonic sensor 232. In some embodiments, the amplifier may amplify signals generated by the binding of a target analyte to a probe molecule, such as but not limited to a binding ligand as described above, which may allow for more precise and accurate detection of the target analyte. In an embodiment, the amplifier may include a biochemical amplifier. For example, and without limitation, the biochemical amplifier may include a nanoparticle such as but not limited to magnetic beads nanoparticles, gold nanoparticles, magnetic nanoparticle, magnetic gold particle, and other magnetic detection particles. As another non-limiting example, the amplifier may include an enzyme, such as but not limited to horseradish peroxidase (HRP) or alkaline phosphatase (ALP). As another non-limiting example, the amplifier may include nucleic acid amplification such as but not limited to Polymerase chain reaction (PCR). In another embodiment, the amplifier may include an electromechanical amplifier. For example, and without limitation, the electromechanical amplifier may include cyclic voltammetry or chronoamperometry. Additional disclosure related to the amplifier may be found in U.S. patent application Ser. No. 18/299,271, filed on May 18, 2023, entitled “APPARATUS AND METHOD FOR DETECTING AN ANALYTE,” having an attorney docket number of 2214-013USU1, the entirety of which is incorporated herein as a reference.

With continued reference to FIGS. 2A-B, an apparatus 200 includes a photonic sensor 232. For the purposes of this disclosure, “photonic sensor” a sensor that includes electronic components that form a functional circuit that detects light. In some embodiments, the photonic sensor 232 is configured to output a sensor signal to a reader device 240 as described below. Additionally and without limitation, the photonic sensor 232 disclosed herein may be consistent with a sensor device found in U.S. patent application Ser. No. 18/221,712, filed on Mar. 15, 2023, entitled as “APPARATUS AND METHODS FOR PERFORMING MICROFLUIDIC-BASED BIOCHEMICAL ASSAYS,” having an attorney docket number of 2214-008USU1, and a photonic sensor chip in U.S. patent application Ser. No. 18/226,014, filed on Mar. 24, 2023, entitled as “PHOTONIC BIOSENSORS FOR MULTIPLEXED DIAGNOSTICS AND A METHOD OF USE,” having an attorney docket number of 2214-010USU1, the entirety of which are incorporated herein by reference.

With continued reference to FIGS. 2A-B, in some embodiments, a photonic sensor 232 may include an optical waveguide. For the purposes of this disclosure, an “optical waveguide” is a structure that is designed to confine and guide electromagnetic waves along a path from one point to another. As a non-limiting example, electromagnetic waves may include ultraviolet, x-rays, gamma rays, infrared, microwave, radio waves, visible light, and the like. In some embodiments, the photonic sensor 232 may include a plurality of optical waveguide. In some embodiments, the optical waveguide may include dielectric materials, silicon, glass, polymer, semiconductor, and the like. In some embodiments, the optical waveguide may include various geometry of the waveguide. As a non-limiting example, the optical waveguide may include a straight waveguide, tapered waveguide, grating waveguide, and the like. In some embodiments, the optical waveguide may include various shapes, such as but not limited to rectangular, circular, elliptical cross-sections, and the like. In some embodiments, the optical waveguide may include optical fiber waveguides, transparent dielectric waveguides, liquid light guides, liquid waveguides, light pipe, laser-inscribed waveguide, and the like. In some embodiments, the optical waveguide may include planar, strip, rib, fiber waveguides, and the like. In some embodiments, the optical waveguide may include single-mode, multi-mode, and the like. In some embodiments, the optical waveguide may include various refractive index distributions such as but not limited to step index distribution, gradient index distribution, and the like. For the purposes of this disclosure, “refractive index” of a material is a measure of how much the material can bend, or refract, light as it passes through it.

With continued reference to FIGS. 2A-B, in some embodiments, an optical waveguide may be configured to output an optical output. For the purposes of this disclosure, an “optical output” is an optical signal that is output from an optical waveguide. As a non-limiting example, the optical waveguide may output the optical output to at least a photodetector. As another non-limiting example, the optical waveguide may output the optical output to a fiber-optic cable. In some embodiments, a design and optimization of the optical waveguides may depend on the wavelength of the optical signal, polarization state, refractive index of materials used and/or mode profile of an output source.

With continued reference to FIGS. 2A-B, a photonic sensor 232 may include one or more resonators. For the purposes of this disclosure, a “resonator” is a structure made of waveguide that can trap, store, transmit, process electromagnetic waves. In an embodiment, the one or more resonators may include photonic crystal cavities, grating structures, or interferometric structures such as Mach-Zehnder or Michelson interferometers, and the like. For the purposes of this disclosure, a “grating structure” is a structure of any regularly spaced collection of essentially identical, parallel, elongated elements, such as but not limited to optical waveguides. A “period Λ” of the grating determines the diffraction. As a non-limiting example, the grating structures may (SWG). For the purposes of this disclosure, a “sub-wavelength include a silicon sub-wavelength grating grating” is grating structures with a period Λ that is sufficiently small compared to the wavelength of light.

With continued reference to FIGS. 2A-B, in some embodiments, one or more resonators may include one or more ring resonators. For the purposes of this disclosure, a “ring resonator” is a waveguide that is a closed loop. In some embodiments, the one or more resonators may include various sizes and shapes of the loop (or a ring) and refractive index. In some embodiments, one or more ring resonators may be coupled with an optical waveguide. As a non-limiting example, one or more ring resonators may be in contact with the optical waveguide. As another non-limiting example, the one or more ring resonators may include a gap between the one or more ring resonators and the optical waveguide. In some embodiments, one or more ring resonators may use the principle of resonant wave coupling to filter or select certain wavelengths of light. In some embodiments, the photonic sensor 232 may include one or more arrays of one or more ring resonators. In an embodiment, each of the one or more ring resonators may detect the same one or more analytes of at least a fluid. In another embodiment, each of the one or more ring resonators may detect different one or more analytes of the at least a fluid. As a non-limiting example, one ring resonator of the one or more ring resonators may detect SARS-COV-protein while another ring resonator of the one or more ring resonators detects glucose. When light is input into the loop (or the ring) of one or more ring resonators, the light may circulate around the loop multiple times due to total internal reflection, creating a standing wave pattern with constructive and/or destructive interference. Then, the one or more resonators may output an optical output. Because only a select few wavelengths are at resonance within the loop of the one or more ring resonators, the one or more ring resonators may function as a filter. In an embodiment, the light (or an input optical signal) may be input to a of an optical waveguide of an optical waveguide, where the light may be from a fiber-optic cable with a PM fiber delivering the light from at least a light source. In another embodiment, an optical output may be output from a of an optical waveguide of the optical waveguide to at least a photodetector. As a non-limiting example, the optical output may be output from the of an optical waveguide of the optical waveguide to the at least a photodetector, then, to a reader device 240.

With continued reference to FIGS. 2A-B, in some embodiments, one or more ring resonators may include a single-ring resonator, double-ring resonator, add-drop filter, Vernier ring resonator, Bragg grating ring resonator, and the like. In some embodiments, one or more ring resonators may include a microring resonator. For the purposes of this disclosure, a “microring resonator” is a miniaturized version of the ring resonator. In some embodiments, the microring resonator may be fabricated with a silicon or silicon-on-insulator (SOI) substrate using photolithography, etching, deposition, and/or other microfabrication techniques. For the purposes of this disclosure, “silicon-on-insulator substrate” is a type of semiconductor substrate. As a non-limiting example, the SOI substrate may include a thin layer of silicon, such as but not limited to silicon dioxide, on top of a layer of insulating material which is itself on top of a bulk silicon substrate. In some embodiments, the SOI substrate may reduce capacitance and parasitic effects, provide better isolation between devices, improve radiation hardness, and the like. In some embodiments, the SOI substrate may be fabricated for optical waveguides, ring resonators, and other photonic structures.

With continued reference to FIGS. 2A-B, in some embodiments, one or more resonators may include a respective layer of binding ligands. For the purposes of this disclosure, a “binding ligand” is a ligand that is capable of binding an analyte. Additional disclosure related to the binding ligand disclosed herein may be found in International Patent Application No. PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to FIGS. 2A-B, in some embodiments, a photonic sensor 232 may utilize evanescent field of an optical waveguide and one or more resonators to probe properties and/or characteristics of the surrounding medium such as but not limited to one or more analytes of at least a fluid. For the purposes of this disclosure, “evanescent field” is a type of electromagnetic field that exists outside the core of an optical waveguide. The evanescent field may decay exponentially with distance from the core and may carry less energy than the propagating mode inside the waveguide. When the waveguide and/or the one or more resonators is brought close to the one or more analytes of the at least a fluid, where the one or more analytes are immobilized on the surface of the waveguide and/or the one or more resonators such as but not limited to with binding ligands, one or more characteristics of the one or more analytes such as but not limited to their concentration, binding kinetics, conformational changes, or the like, may be probed using the evanescent field. Additional disclosure related to various methods to sense the one or more analytes may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to FIGS. 2A-B, in some embodiments, a photonic sensor 232 may include at least a photodetector. In some cases, the photonic sensor 232 may include a plurality of photodetectors, for instance a first photodetector and a second photodetector. In some cases, the first photodetector and/or the second photodetector may be configured to measure one or more of first optical output and second optical output, from a first waveguide and a second waveguide, respectively, such as but not limited to an of an optical waveguide. The at least a first photodetector may be configured to convert the first optical output into a first sensor signal as a function of variance of an optical property of the first waveguide, where the first sensor signal may include without limitation any voltage and/or current waveform. Additionally, or alternatively, the photonic sensor 232 may include a second photodetector located down beam from the second waveguide. In some embodiments, the second photodetector may be configured to measure a variance of an optical property of second waveguide and convert the second optical output into a second sensor signal as a function of the variance of the optical property of the second waveguide.

With continued reference to FIGS. 2A-B, as used in this disclosure, a “photodetector” is any device that is sensitive to light and thereby able to detect light. In some cases, the at least a photodetector may include a photodiode, a photoresistor, a photosensor, a photovoltaic chip, and the like. In some cases, the at least a photodetector may include a Germanium-based photodiode. The at least a photodetector may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. “Avalanche Photo Diodes (APDs),” as used herein, are diodes (e.g., without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs may provide a built-in stage of gain through avalanche multiplication. When the reverse bias is less than the breakdown voltage, the gain of the APD may be approximately linear. For silicon APDs, this gain may be on the order of 10-200. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage may be referred to as a Single Photon Avalanche Diode, or SPAD. In this case, the n-p electric field may be sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode.

With continued reference to FIGS. 2A-B, in some cases, at least a photodetector may include a photosensor array, for example without limitation a one-dimensional array. The photosensor array may be configured to detect a variance in an optical property of waveguide. In some cases, first photodetector and/or second photodetector may be wavelength dependent. For instance, and without limitation, first photodetector and/or second photodetector may have a narrow range of wavelengths to which each of first photodetector and second photodetector are sensitive. As a further non-limiting example, each of first photodetector and second photodetector may be preceded by wavelength-specific optical filters such as bandpass filters and/or filter sets, or the like; in any case, a splitter may divide output from optical matrix multiplier as described below and provide it to each of first photodetector and second photodetector. Alternatively, or additionally, one or more optical elements may divide output from waveguide prior to provision to each of first photodetector and second photodetector, such that each of first photodetector and second photodetector receives a distinct wavelength and/or set of wavelengths. For example, and without limitation, in some cases a wavelength demultiplexer may be disposed between waveguides and first photodetector and/or second photodetector; and the wavelength demultiplexer may be configured to separate one or more lights or light arrays dependent upon wavelength. As used in this disclosure, a “wavelength demultiplexer” is a device that is configured to separate two or more wavelengths of light from a shared optical path. In some cases, a wavelength demultiplexer may include at least a dichroic beam splitter. In some cases, a wavelength demultiplexer may include any hot mirror, a cold mirror, a short-pass filter, a long pass filter, a notch filter, and the like. An exemplary wavelength demultiplexer may include part No. WDM-11P from OZ Optics of Ottawa, Ontario, Canada. Further examples of demultiplexers may include, without limitation, gratings, prisms, and/or any other devices and/or components for separating light by wavelengths that may occur to persons skilled in the art upon reviewing the entirety of this disclosure. In some cases, at least a photodetector may be communicative with computing device, such that a sensed signal such as but not limited to one or more sensor signals may be communicated with computing device of a reader device 240.

With continued reference to FIGS. 2A-B, an apparatus 200 includes a reader device 240. For the purposes of this disclosure, a “reader device” is a device that processes signals for, generated by, or received by a photonic sensor. As a non-limiting example, the reader device 240 is configured to detect one or more characteristics of extracted fluid as a function of a sensor signal. For the purposes of this disclosure, a “characteristic” of a fluid is a distinguishing feature of a fluid. As a non-limiting example, the one or more characteristics may include presences of one or more analytes in the at least a fluid, concentration level of the one or more analytes in the at least a fluid, binding kinetics, conformational changes, and the like. Additional disclosure related to the one or more characteristics of the fluid and/or the methods to determine the one or more characteristics of the fluid may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein as a reference. The reader device 240 may be communicatively connected to the photonic sensor 232. In some embodiments, the reader device 240 may be connected with the photonic sensor 232 using a connecting system as described below. For the purposes of this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIGS. 2A-B, a reader device 240 may include a computing device. In some embodiments, a processor and a memory communicatively connected to the processor may be included in the computing device. The computing device may include any computing device as described in this disclosure, including without limitation a microcontroller, microprocessor, digital signal processor (DSP) and/or system on a chip (SoC) as described in this disclosure. The computing device may include, be included in, and/or communicate with a mobile device such as a mobile telephone or smartphone. The computing device may include a single computing device operating independently, or may include two or more computing device operating in concert, in parallel, sequentially or the like; two or more computing devices may be included together in a single computing device or in two or more computing devices. The computing device may interface or communicate with one or more additional devices as described below in further detail via a network interface device. Network interface device may be utilized for connecting the computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. The computing device may include one or more computing devices dedicated to data storage, security, distribution of traffic for load balancing, and the like. the computing device may distribute one or more computing tasks as described below across a plurality of computing devices of computing device, which may operate in parallel, in series, redundantly, or in any other manner used for distribution of tasks or memory between computing devices. the computing device may be implemented, as a non-limiting example, using a “shared nothing” architecture.

With continued reference to FIGS. 2A-B, a computing device may be designed and/or configured to perform any method, method step, or sequence of method steps in any embodiment described in this disclosure, in any order and with any degree of repetition. For instance, the computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIGS. 2A-B, in some embodiments, a reader device 240 may include one or more elements of dedicated signal processing hardware and/or software modules. This may include filters, filter banks including analysis and synthesis banks, fast Fourier Transform (FFT) calculation modules, signal generators, matrix operation calculators, or the like. In some embodiments, the reader device 240 may be configured to perform one or more signal processing steps on a signal, where the signal is any signal disclosed in the entirety of this disclosure. As used in this disclosure, a “signal” is any intelligible representation of data, for example from one device to another. A signal may include an optical signal, a hydraulic signal, a pneumatic signal, a mechanical signal, an electric signal, a digital signal, an analog signal, and the like. In some cases, a signal may be used to communicate with a computing device, for example by way of one or more ports. In some cases, a signal may be transmitted and/or received by a computing device, for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 228 [printer port]), and the like.

With continued reference to FIGS. 2A-B, as a non-limiting example, a reader device 240 may analyze, modify, and/or synthesize a signal representative of characteristic. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which may vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued, and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. In some embodiments, a filter bank may be used, such as but not limited to analysis banks, synthesis banks, FFT filter banks, and the like. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIGS. 2A-B, a reader device 240 may include at least a light source. The reader device 240 may be configured to provide an input optical signal using at least one light source. For the purposes of this disclosure, an “input optical signal” is an optical signal that includes electromagnetic radiation. In some embodiments, the input optical signal may be transmitted over optical fibers. As used in this disclosure, a “light source” is any device configured to emit electromagnetic radiation. As a non-limiting example, electromagnetic radiation may include ultraviolet (UV), visible light, infrared light, and the like. At least a light source may control propagation, direction, polarization, intensity of light waves. In some embodiments, the photonic sensor 232 may include lenses, mirrors, prisms, filters, optical fibers, and the like. In some embodiments, at least a light source may be tuned across the resonances of elements of a photonic sensor 232. In some cases, at least a light source may include a coherent light source, which is configured to emit coherent light, for example a laser. In some cases, at least a light source may include a non-coherent light source configured to emit non-coherent light, for example a light emitting diode (LED). In some cases, at least a light source may emit a light having substantially one wavelength. In some cases, the at least a light source may emit the light having a wavelength range. The light may have a wavelength in an ultraviolet range, a visible range, a near-infrared range, a mid-infrared range, and/or a far-infrared range. For example, in some cases the light may have a wavelength within a range from about 200 nm to about 20 micrometers. In some cases, the light may have a wavelength within a range of about 400 nm to about 2,500 nm. The at least a light source may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. At least a light source may additionally or alternatively include a light-emitting diode (LED), an organic LED (OLED) and/or any other light emitter. In some cases, at least a light source may be configured to couple light into a photonic sensor 232 for instance into one or more waveguide described above.

With continued reference to FIGS. 2A-B, a reader device 240 may be configured to receive one or more sensor signals from a photonic sensor 232. For the purposes of this disclosure, a “sensor signal” is a signal obtained from a photonic sensor that is related to one or more analytes of at least a fluid. As a non-limiting example, the sensor signal may include an optical signal, electronic signal, and the like. For example and without limitation, the optical signal may include an optical output from a of an optical waveguide of an optical waveguide. For another example and without limitation, the electronic signal may include a signal from at least a photodetector of the photonic sensor 232. As another non-limiting example, one or more sensor signals may include a resonance wavelength shift. For the purposes of this disclosure, a “resonance wavelength shift” is a change in resonant wavelength of a photonic sensor, such as but not limited to a ring resonator, due to one or more analytes of at least a fluid.

With continued reference to FIGS. 2A-B, in some embodiments, a reader device 240 may receive one or more sensor signals from a photonic sensor 232 as the reader device 240 provides an input optical signal to the photonic sensor 232. In an embodiment, the reader device 240 may receive the one or more sensor signals, such as but not limited to an output signal, from an optical waveguide, such as but not limited to a of an optical waveguide of the photonic sensor 232 using an optical fiber. In another embodiment, the reader device 240 may receive one or more sensor signals from at least a photodetector of the photonic sensor 232. As a non-limiting example, the optical fiber and/or the at least a photodetector may receive the output signal using a vertical coupling, edge coupling, a grating coupler, or any couplers thereof. Additionally, vertical coupling, edge coupling, the grating coupler may be implemented in any other components of the photonic sensor 232 and the reader device 240.

With continued reference to FIGS. 2A-B, a reader device 240 is configured to determine one or more characteristics of one or more analytes of at least a fluid as a function of one or more sensor signals. In some embodiments, the reader device 240 may determine one or more characteristics of the one or more analytes of the at least a fluid as the reader device 240 received one or more sensor signals from the photonic sensor 232. In some embodiments, the reader device 240 may include at least a photodetector. At least a photodetector of the reader device 240 disclosed herein may be consistent with any photodetectors described in the entirety of this disclosure. As a non-limiting example, when the reader device 240 receives an optical output, the at least a photodetector of the reader device 240 may receive the optical output and convert the optical output to one or more sensor signals to determine the one or more characteristics of the one or more analytes of the at least a fluid. As a non-limiting example, the one or more characteristics of the one or more analytes of the at least a fluid may include presences of one or more analytes in the at least a fluid, concentration level of the one or more analytes in the at least a fluid, and the like. The one or more characteristics of the one or more analytes may be determined as a function of a change in a resonance wavelength, change in optical wavelength, change of concentration level, and the like. For example and without limitation, a shift in the resonance wavelength of one or more resonators may indicate the presence of the one or more analytes of interest. Additional disclosure related to the one or more characteristics of the one or more analytes and/or the methods to determine the one or more characteristics of the one or more analytes may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein as a reference.

With continued reference to the FIGS. 2A-B, an apparatus 200 includes an assay component 216. For the purposes of this disclosure, an “assay component” is a component used in the analysis of fluids. In some embodiments, the assay component 216 is fluidically connected to a microfluidic assembly 240 as described above. As a non-limiting example, the assay component 216 is configured to test the extracted fluid to detect one or more characteristics of the extracted fluid. In some embodiments, the assay component 216 may include a lateral flow assay. For the purposes of this disclosure, a “lateral flow assay” is a type of diagnostic test that detects the presence or absence of a target substance in a sample that migrates parallel to a surface on an assay component. In some embodiments, the lateral flow assay may include a paper-based strip. As a non-limiting example, extracted fluid can be applied to one end of the paper-based strip and then migrates along the paper-based strip by capillary action. As the extracted fluid flows through the paper-based strip, it may pass through different zones that include capture molecules that are specific to the target substance (e.g., analyte). For example, and without limitation, the capture molecules may include antibodies, DNA probes, binding ligands, and the like. If the target substance is present in the sample, the target substance may bind to the capture molecules and create a visible signal, such as a colored line, in the detection zone of the paper-based strip. In some embodiments, the assay component 216 may be used for point-of-care testing. For the purposes of this disclosure, “point-of-care diagnostic” is a technique of diagnosis that allows detection and diagnosis of diseases at or near the patient site. For the purposes of this disclosure, a “patient site” is a patient's physical location at the time of receipt of a sample from the patient's body. In some embodiments, the assay component 216 may be configured for a multiplexed diagnostic. For the purposes of this disclosure, “multiplexed diagnostic” is a technique of diagnosis that can detect multiple analytes in a single sample.

With continued reference to the FIGS. 2A-B, in some embodiments, a photonic sensor 232 may be further configured to detect a change in an assay component 216. As a non-limiting example, the photonic sensor 232 may detect a change in a lateral flow assay by using light to measure the intensity of the color that develops in the test line (a color line of the lateral flow assay). When extracted fluid is applied to a paper-based strip, the extracted fluid may flow through the test line where it interacts with specific reagents. As a color change occurs in the test line due to the binding of the analyte and the reagents, the photonic sensor 232 may emit light at a certain wavelength and may measure the intensity of the light that is transmitted through the test line. If a color change has occurred, the intensity of the transmitted light will be different, indicating the presence of the target analyte. This change in light intensity can be detected and processed by the photonic sensor 232 to generate a sensor signal to output to a reader device 240. Then the reader device 240 may analyze the sensor signal to determine characteristics of the extracted fluid as a function of the sensor signal.

With continued reference to the FIGS. 2A-B, in some embodiments, an assay component 216 may include a test timing feature. For the purposes of this disclosure, a “test timing feature” is a feature of an assay component that indicates when the test is complete or when it is time to read the results. As a non-limiting example, the test timing feature may include an indicator that indicates to a user that the test is complete or it is time to read the results. As another non-limiting example, the indicator may indicate to the user that the assay component 216 can be removed from a housing. As another non-limiting example, the test timing feature may include an indicator that indicates a photonic sensor 232 that the test is complete. An exemplary configuration of the test timing feature of the assay component is shown in FIG. 7.

With continued reference to the FIGS. 2A-B, an apparatus 200 includes a fluid collecting reservoir 224. For the purposes of this disclosure, a “fluid collecting reservoir” is a component that is configured to collect a fluid. In some embodiments, the fluid collecting reservoir 224 is fluidically connected to the microfluidic assembly. As a non-limiting example, the fluid collecting reservoir 224 is configured to collect extracted fluid from a microfluidic assembly 240. As another non-limiting example, the fluid collecting reservoir 224 may be further configured to passively pump the extracted fluid for the flow of the extracted fluid of the microfluidic assembly 240 as described above. In some embodiments, the fluid collecting reservoir 224 may include a fluid collection pad. For the purposes of this disclosure, a “fluid collection pad” is a material or device designed to collect and absorb fluids. In some embodiments, the fluid collection pad may include materials that absorb the fluids. As a non-limiting example, the fluid collection pad may include cotton, cellulose, or other absorbent polymers. In some embodiments, the fluid collecting reservoir 224 may include a fluid collecting container. As another non-limiting example, the fluid collecting reservoir 224 may include a microtiter tube, a dried blood spot, a plasma separating paper, and the like. In some embodiments, the fluid collecting container may include materials that are compatible with the fluids. As a non-limiting example, the fluid collecting container may include plastic, glass, and the like. In some embodiments, the fluid collecting container may include anticoagulants or preservatives to prevent the blood sample from clotting or deteriorating during storage and transportation. In some embodiments, the fluid collecting reservoir 224 may be removably inserted into a housing as described above. Once, in a non-limiting example, the fluid collecting reservoir 224 is removed from the housing, the fluid collecting reservoir 224 can be sent using a mailing system for further testing. In some embodiments, the fluid collecting reservoir 224 may be disposable. In some embodiments, the fluid collecting reservoir 224 may be replaceable. In some embodiments, the fluid collecting reservoir 224 may be used for laboratory-based diagnostic. As a non-limiting example, the fluid collecting reservoir 224 may be sent to a laboratory for diagnostic purposes. For example, and without limitation, the fluid collecting reservoir 224 may be mailed to the laboratory and analyzed in the laboratory by a trained personnel. In some embodiments, inside the fluid collecting reservoir 224, the collected fluid may be separated into different streams going to the different immediate test areas and the different storage techniques. In some embodiments, the collected fluid may be either diluted, undiluted or lysed.

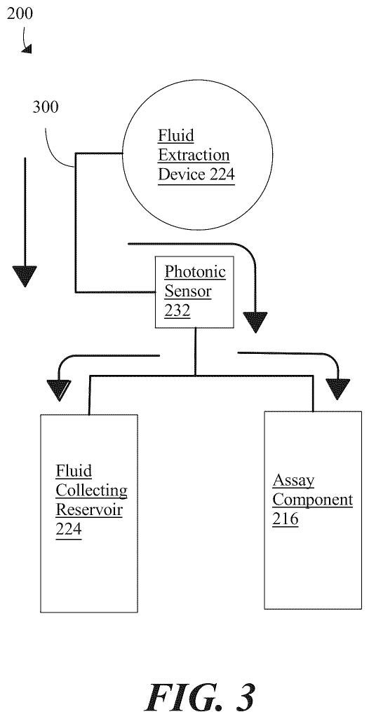

Referring now to FIG. 3, an exemplary workflow of an apparatus 300 of a multipurpose fluid extraction device for diagnostics is illustrated. In some embodiments, a photonic sensor 332 may output a sensor signal to the reader device 340. The sensor signal may be enabled by ring resonators and a functionalized surface of the photonic sensor 332 that can facilitate specific binding. For an amplified binding, amplifiers can be placed on microfluidic channels 300 of a microfluidic assembly 340 to later be driven alongside extracted fluid onto the photonic sensor 332. The amplified sensor signal may be reported on/with the reader device 340.

Other embodiments of assay and collection devices may include embodiments as disclosed in U.S. Nonprovisional application Ser. No. 18/121,712, filed on Mar. 15, 2023, and entitled “APPARATUS AND METHODS FOR PERFORMING MICROFLUIDIC-BASED BIOCHEMICAL ASSAYS” the entirety of which is incorporated herein by reference.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

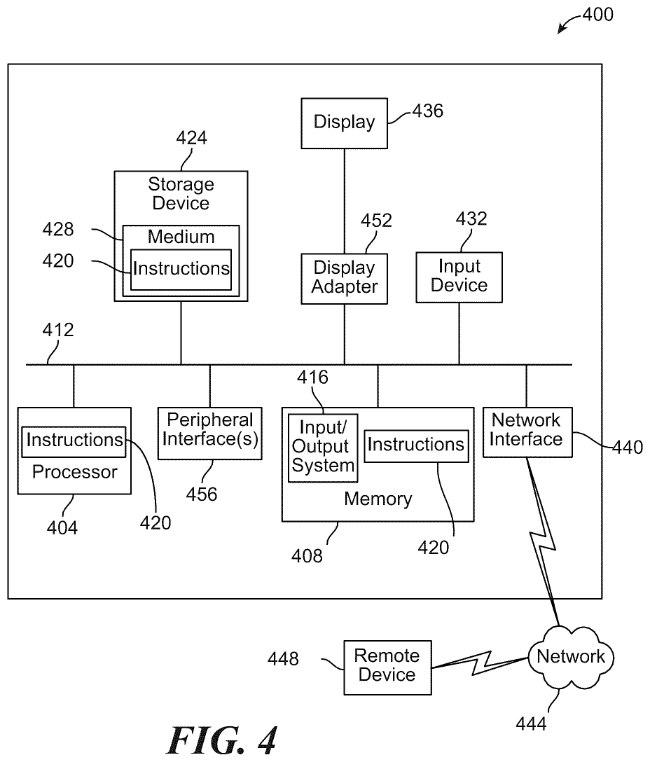

FIG. 4 shows a diagrammatic representation of one embodiment of a computing device in the exemplary form of a computer system 400 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 400 includes a processor 404 and a memory 408 that communicate with each other, and with other components, via a bus 412. Bus 412 may include any of several types of bus structures including, but not limited to, a memory bus, a memory controller, a peripheral bus, a local bus, and any combinations thereof, using any of a variety of bus architectures.

Processor 404 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 404 may be organized according to Von Neumann and/or Harvard architecture as a non-limiting example. Processor 404 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 408 may include various components (e.g., machine-readable media) including, but not limited to, a random-access memory component, a read only component, and any combinations thereof. In one example, a basic input/output system 416 (BIOS), including basic routines that help to transfer information between elements within computer system 400, such as during start-up, may be stored in memory 408. Memory 408 may also include (e.g., stored on one or more machine-readable media) instructions (e.g., software) 420 embodying any one or more of the aspects and/or methodologies of the present disclosure. In another example, memory 408 may further include any number of program modules including, but not limited to, an operating system, one or more application programs, other program modules, program data, and any combinations thereof.

Computer system 400 may also include a storage device 424. Examples of a storage device (e.g., storage device 424) include, but are not limited to, a hard disk drive, a magnetic disk drive, an optical disc drive in combination with an optical medium, a solid-state memory device, and any combinations thereof. Storage device 424 may be connected to bus 412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 424 (or one or more components thereof) may be removably interfaced with computer system 400 (e.g., via an external port connector (not shown)). Particularly, storage device 424 and an associated machine-readable medium 428 may provide nonvolatile and/or volatile storage of machine-readable instructions, data structures, program modules, and/or other data for computer system 400. In one example, software 420 may reside, completely or partially, within machine-readable medium 428. In another example, software 420 may reside, completely or partially, within processor 404.

Computer system 400 may also include an input device 432. In one example, a user of computer system 400 may enter commands and/or other information into computer system 400 via input device 432. Examples of an input device 432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 432 may be interfaced to bus 412 via any of a variety of interfaces (not shown) including, but not limited to, a serial interface, a parallel interface, a game port, a USB interface, a FIREWIRE interface, a direct interface to bus 412, and any combinations thereof. Input device 432 may include a touch screen interface that may be a part of or separate from display 436, discussed further below. Input device 432 may be utilized as a user selection device for selecting one or more graphical representations in a graphical interface as described above.

A user may also input commands and/or other information to computer system 400 via storage device 424 (e.g., a removable disk drive, a flash drive, etc.) and/or network interface device 440. A network interface device, such as network interface device 440, may be utilized for connecting computer system 400 to one or more of a variety of networks, such as network 444, and one or more remote devices 448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 444, may employ a wired and/or a wireless mode of communication. In general, any network topology may be used. Information (e.g., data, software 420, etc.) may be communicated to and/or from computer system 400 via network interface device 440.

Computer system 400 may further include a video display adapter 452 for communicating a displayable image to a display device, such as display device 436. Examples of a display device include, but are not limited to, a liquid crystal display (LCD), a cathode ray tube (CRT), a plasma display, a light emitting diode (LED) display, and any combinations thereof. Display adapter 452 and display device 436 may be utilized in combination with processor 404 to provide graphical representations of aspects of the present disclosure. In addition to a display device, computer system 400 may include one or more other peripheral output devices including, but not limited to, an audio speaker, a printer, and any combinations thereof. Such peripheral output devices may be connected to bus 412 via a peripheral interface 456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A bodily fluid collection assembly, the assembly comprising:
a collection device comprising:
a tube comprising a chemical lining; and
a cap;
a multipurpose fluid extraction device attached to the collection device, the multipurpose fluid extraction device comprising:
a fluid extraction system, wherein the fluid extraction system is configured to extract a fluid from a user;
a microfluidic assembly, wherein the microfluidic assembly is configured to provide a flow of extracted fluid and wherein the microfluidic assembly comprises a microfluidic channel;
an assay component fluidically connected to the microfluidic assembly, wherein the assay component is configured to test the extracted fluid; and
a fluid collecting reservoir fluidically connected to the microfluidic assembly, wherein the fluid collecting reservoir is configured to collect the extracted fluid from the microfluidic assembly.

2. The assembly of claim 1, wherein the tube comprises an outer body, wherein the outer body comprises a plurality of ridges projecting from the outer body.

3. The assembly of claim 1, wherein the collection device further comprises a fastener connecting the cap to the tube.

4. The assembly of claim 1, wherein the chemical lining comprises a temperature-stabilizing gel.

5. The assembly of claim 1, wherein the chemical lining comprises an anticoagulant coating.

6. The assembly of claim 1, wherein the chemical lining comprises a chemical stabilizer.

7. The assembly of claim 1, wherein the collection device is removably inserted into an aperture of the multipurpose fluid extraction device.

8. The assembly of claim 1, wherein a distal end of the tube comprises a malleable material and is configured to cause ejection of fluid from the tube when the tube is squeezed.

9. The assembly of claim 1, wherein the cap comprises a push-on cap.

10. The assembly of claim 1, wherein the cap comprises a distal portion comprising an opening, wherein the opening is configured to allow fluid to flow through it.

11. The assembly of claim 1, further comprising a housing configured to encapsulate at least a portion of the multipurpose fluid extraction device, wherein the housing is portable.

12. The assembly of claim 11, wherein the housing comprises a first housing and second housing, the second housing is placed on atop of the first housing.

13. The assembly of claim 11, wherein the assay component is configured to be removably inserted into the housing through a first aperture of the housing.

14. The assembly of claim 11, wherein the fluid collecting reservoir is configured to be removably inserted into the housing through a second aperture of the housing.

15. The assembly of claim 1, wherein the microfluidic assembly is further configured to provide the flow of the extracted fluid over a photonic sensor.

16. The assembly of claim 1, wherein the fluid collecting reservoir is further configured to passively pump the extracted fluid for the flow of the extracted fluid of the microfluidic assembly.

17. The assembly of claim 1, wherein the microfluidic assembly further comprises a photonic sensor, wherein the photonic sensor is configured to output a sensor signal to a reader device.

18. The assembly of claim 18, wherein the photonic sensor comprises a microring resonator.

19. The assembly of claim 1, wherein the microfluidic assembly further comprises a reader device, wherein the reader device is configured to detect one or more characteristics of the extracted fluid as a function of the sensor signal.

20. The assembly of claim 1, wherein one or more characteristics of the extracted fluid comprises a concentration level of one or more analytes in the extracted fluid.
